(19) United States
(12) Patent Application Publication
(10) Pub. No.: US 2024/0156406 A1
(43) Pub. Date: May 16, 2024

(54) SYSTEMS AND METHODS FOR MEASURING EXTRAVASCULAR LUNG WATER

(71) Applicant: Shifamed Holding, LLC, Campell, CA (US)

(72) Inventors: Peter Andriola, Castro Valley, CA (US); Brian Fahey, Menlo Park, CA (US)

(21) Appl. No.: 18/551,930

(22) PCT Filed: Apr. 22, 2022
(86) PCT No.: PCT/US2022/025923

§ 371 Date: Sep. 22, 2023

Related U.S. Application Data
(60) Provisional application No. 63/178,715, filed on Apr. 23, 2021.

(51) Int. Cl. A61B 5/00; A61B 5/0537; A61B 5/145

(52) U.S. Cl. CPC A61B 5/686; A61B 5/0537; A61B 5/14546

(57) ABSTRACT

The present technology is directed to measuring, estimating, or otherwise determining extravascular lung water (EVLW) in a patient. The systems can include a first device configured to be implanted in the patient and a second device configured to remain external to the patient. The first device can transmit electromagnetic energy, receive electromagnetic energy, or both transmit and receive electromagnetic energy. Likewise, the second device can receive electromagnetic energy, transmit electromagnetic energy, or both receive and transmit electromagnetic energy. In operation, an electromagnetic energy transmission pathway between the first device and the second device can include the patient's lungs. Accordingly, the system can determine EVLW in the patient based at least in part on an attenuation or phase shift of electromagnetic energy transmitted between the first device and the second device along the transmission path.

### CROSS-REFERENCE TO RELATED APPLICATION(S)

This application claims the benefit of U.S. Provisional Patent Application No. 63/178,715, filed Apr. 23, 2021, and incorporated by reference herein by reference in its entirety.

### TECHNICAL FIELD

The present technology is generally directed to systems and methods for measuring extravascular lung water and other physiologic parameters that may, at least in part, be determined by variations in the human body's electromagnetic properties, such as hydration status, cardiac output, or pulmonary tidal volume.

### BACKGROUND

The healthy lung is about 80% water, with the gas exchange surface of the alveolar membrane protected by various barriers and drains. In multiple disease states, injury or elevated pulmonary pressures can cause these protective mechanisms to fail, resulting in the abnormal accumulation of extravascular lung water (EVLW) and alveolar flooding, with consequent loss of respiratory capacity. These disease states include, but are not limited to, heart failure, sepsis, and acute respiratory distress syndrome (ARDS). In heart failure patients, for example, elevated ventricular filling pressures leading to pulmonary congestion produce worsening symptoms of dyspnea that often require hospitalization for management.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIG. 1 is a partially schematic illustration of a system 100 for measuring extravascular lung water in a patient and configured in accordance with select embodiments of the present technology.

FIG. 2A is a schematic illustration of an interatrial device implanted in a heart and configured in accordance with select embodiments of the present technology.

FIG. 2B is a schematic illustration of an interatrial shunting system configured in accordance with select embodiments of the present technology.

### DETAILED DESCRIPTION

The present technology is directed to measuring, estimating, or otherwise determining extravascular lung water (EVLW) in a patient. In some embodiments, the systems can include a first device configured to be implanted in the patient and a second device configured to remain external to the patient. The first device can be configured to transmit electromagnetic energy to the second device, receive electromagnetic energy transmitted from the second device, or both receive electromagnetic energy from and transmit electromagnetic energy to the second device. Likewise, the second device can be configured to receive electromagnetic energy transmitted by the first device, transmit electromagnetic energy to the first device, or both receive electromagnetic energy from and transmit electromagnetic energy to the first device. The second device may be comprised of a single RF transceiving component or a multiplicity of spatially diverse RF transceiving components. In use, an electromagnetic energy transmission path can be formed between the first device and the second device, and this path can include at least a portion of the patient's lungs. The system can therefore determine EVLW in the patient, for example, based at least in part on an attenuation and/or phase shift of electromagnetic energy transmitted between the first device and the second device along the transmission path, or based upon another property or characteristic of the transmitted and/or received signal(s).

Other physiologic parameters such as hydration status, cardiac output, and/or pulmonary tidal volume have similar effects on attenuation and phase shift of electromagnetic energy transmitted between the first device and the second device along the transmission path, and can be distinguished from each other based on time scale. For example, EVLW can create changes in these electromagnetic parameters on a timescale of hours-days, hydration status on a timescale of minutes-hours, cardiac output on a time scale seconds, and/or tidal volume on a time scale of 10 s of seconds.

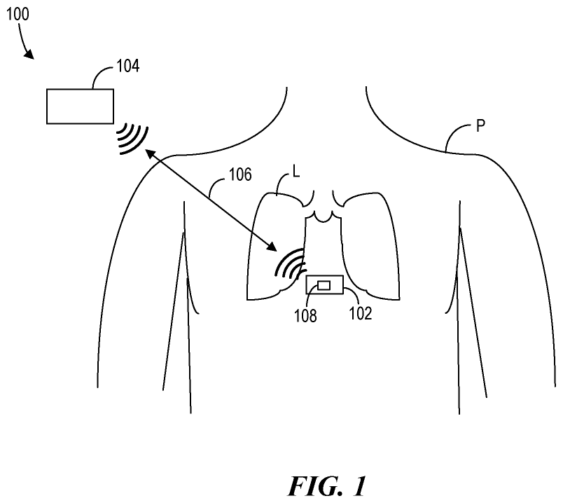

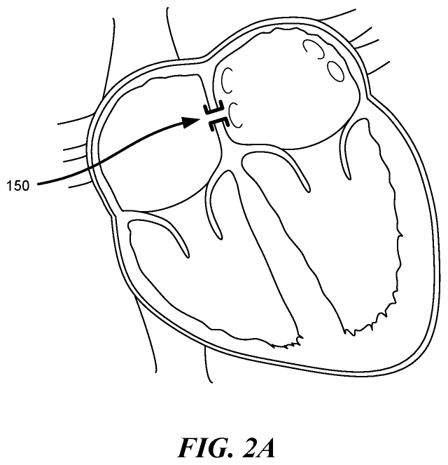

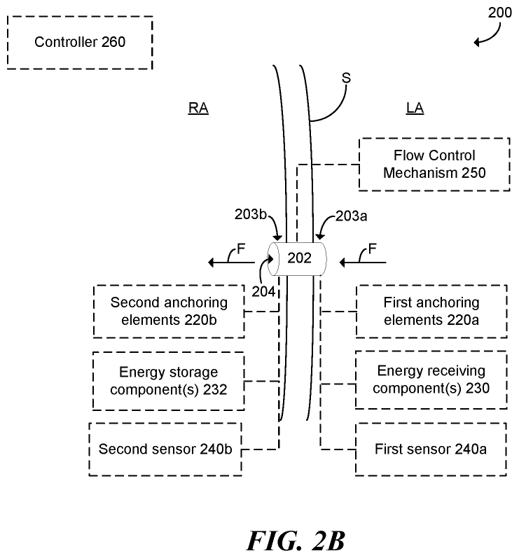

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the claims but are not described in detail with respect to FIGS. 1-2B.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “about” and “approximately” are used herein to mean the stated value plus or minus 10%.

As used herein, the terms “interatrial device,” “interatrial shunt device,” “IAD,” “IASD,” “interatrial shunt,” and “shunt” are used interchangeably to refer to a device that, in at least one configuration, includes a shunting element that provides a blood flow between a first region (e.g., a LA of a heart) and a second region (e.g., a RA or coronary sinus of the heart) of a patient. Although described in terms of a shunt between the atria, namely the LA and the RA, one will appreciate that the technology may be applied equally to devices positioned between other chambers and passages of the heart, between other parts of the cardiovascular system, or between other parts of the body. For example, any of the shunts described herein, including those referred to as “interatrial,” may be nevertheless used and/or modified to shunt between the LA and the coronary sinus, or between the right pulmonary vein and the superior vena cava. Moreover, while applications of the disclosure herein primarily describe medical devices for shunting blood in the heart, the present technology can be readily adapted for medical devices to shunt other fluids—for example, devices used for aqueous shunting, or cerebrospinal fluid shunting. The present technology may also be adapted to a variety of implanted medical devices in addition to shunts. For example, the present technology may improve the functionality of self-guided and/or steerable devices (e.g., catheters), by reducing the cross-sectional size of electrical components and/or reducing power requirements of the device.

The headings provided herein are for convenience only and do not interpret the scope or meaning of the claimed present technology.

### A. Clinical Need for Measuring Extravascular Lung Water (EVLW)

The ability to measure EVLW in everyday settings could be valuable in reducing heart failure hospitalizations. For example, regularly or semi-regularly measuring EVLW may detect increases in EVLW early when the condition may be treatable with outpatient methods and before the situation deteriorates to a state at which the patient requires advanced levels of care. Additionally, routine or regular measurements of EVLW can be a useful diagnostic that can assist the therapeutic management of acute or chronic heart failure or other conditions (e.g., to guide the dosing of a pharmaceutical regimen, to guide adjustment of an implanted medical device, etc.).

Currently utilized methods of estimating EVLW are largely limited to in-hospital use. These include chest radiography, computed tomography, nuclear magnetic resonance (NMR) imaging, positron emission tomography (PET) imaging, trans-pulmonary thermo-dilution (TPTD), lung ultrasound, and bioimpedance tomography. In addition to requiring highly specialized professional equipment, these methods have additional disadvantages, which can include radiation dose, the need for specialized facilities, and confounding of the EVLW measurement by other physiological parameters such as body mass or hydration status.

While other techniques exist to estimate EVLW, these other techniques also have several disadvantages. For example, implanted intrathoracic impedance monitors (e.g., as implemented as a secondary function of a pacemaker, ICD, or other CRM device), have been used for chronic EVLW estimation, but studies have cast doubt on how accurately these estimations track actual EVLW changes and the corresponding ability to use such estimates to guide clinical care and decision making. Recently, a method known as remote dielectric sensing has been introduced for estimating EVLW. This technique uses the properties of reflected electromagnetic radiation in the UHF (ultra-high frequency) and SHF (super-high frequency) bands to estimate EVLW. More specifically, this approach utilizes the unique dielectric properties of water as compared to other tissue constituents, and their influence on absorption and reflection of electromagnetic energy in these bands, to estimate EVLW. While reflection-based remote dielectric sensing has been shown to be a useful diagnostic both in and outside of the healthcare setting, it has the disadvantages of requiring use of an external transmitter unit and an external receiver unit that must be held in precise alignment, sometimes for extended periods of time (up to several hours), during each measurement. In practice, to take measurements during routine outpatient use, this technique would require a compliant patient who is willing to interface routinely (e.g., daily) with the external transmitter unit and the external receiver unit and follow the necessary protocols that are required for accurate EVLW estimation.

Accordingly, there is a need for improved devices and methods for acute or chronic monitoring of EVLW or changes to EVLW. As described in detail below, the present technology provides systems and methods for measuring, estimating, calculating, or otherwise determining EVLW. Moreover, the present technology is expected to provide a clinically-useful degree of accuracy while also requiring a negligible degree of patient interaction. Such systems and methods are expected to improve the accuracy, repeatability, and/or the consistency of measurements when compared with existing approaches that are described in the art.

### B. Systems and Methods for Measuring EVLW

FIG. 1 is a partially schematic illustration of a system 100 for measuring EVLW in a patient P and configured in accordance with select embodiments of the present technology. The system 100 can include a plurality of devices, including a first device 102 (hereinafter referred to as “the implanted device 102”) implanted or configured to be implanted in the patient P and a second device 104 (hereinafter referred to as “the external device 104”) external to the patient P. The implanted device 102 can transmit electromagnetic energy to and/or toward the external device 104 along a transmission path 106. Alternatively, or additionally, the external device 104 can transmit electromagnetic energy to and/or toward the implanted device 102 along the transmission path 106. Accordingly, in some embodiments the implanted device 102 is configured as a transmitter, a receiver, or both a transmitter and receiver. Likewise, the external device 104 can be configured as a transmitter, a receiver, or both a transmitter and receiver.

The system 100 uses electromagnetic energy transmitted between the implanted device 102 and the external device 104 to monitor EVLW or changes in EVLW. An electromagnetic radiation signal that passes through a medium can become attenuated (i.e., its signal strength reduced in magnitude). Because the location of the implanted device 102 can be known with respect to lungs L of the patient P, the transmission path 106 for an electromagnetic radiation signal, and therefore the attenuating media through which the signal passes, is at least partially known. For example, when the patient P is in a given position relative to the external device 104, the amount of lung tissue in the transmission path 106 between the implanted device 102 and the external device 104 can be known or estimated with sufficient precision. To further aid in determining the transmission path 106, the external device 104 can be located such that the patient P is expected to interface with it in a consistent, repeatable manner (e.g., in a pad underneath a mattress). Accordingly, the transmission path 106 for the electromagnetic radiation signal between the implanted device 102 and the external device 104 can be approximately known based on an expected position of the patient P (e.g., the patient laying on the mattress).

Assessments of attenuation in an electromagnetic radiation signal can be implemented to measure or estimate EVLW or changes in EVLW because the presence of water is known to contribute to signal attenuation for certain types of signals. For example, the system 100 could be expecting to measure a certain baseline degree of attenuation between a transmitted signal strength (which is known) and a received signal strength (which is measured) for a given transmission path 106. The expected baseline degree of attenuation could be a function of the signal frequency, the distance between the implanted device 102 and the external device 104, the media along the signal transmission pathway 106, and other factors. If the detected attenuation during a transmission is greater than expected (e.g., is larger than recent transmissions under similar conditions), it can be indicative of water starting to accumulate in the transmission path 106 (and therefore may be indicative of water starting to accumulate in the lungs L).

In some embodiments, the system 100 utilizes (e.g., transmits, receives, etc.) two or more signals operating at different frequencies and that are expected to be affected differently by the presence of water (e.g., experience different levels of attenuation). The two or more signals can be transmitted along the same or substantially the same transmission path 106 through the lungs L, other tissue, and the environment. In some embodiments, a first signal is transmitted at a frequency at which little to no attenuation resulting from the presence of water is expected, and a second signal is transmitted at a frequency at which attenuation resulting from the presence of water is expected. The ratio of the detected signal attenuation of the first and second signals can therefore be used (either directly or as an input into a more complex computation) as an indicator that can be used to estimate EVLW and/or changes in EVLW. In an example embodiment, the amount of attenuation observed in both the first and the second signal can be attributed to factors other than the presence or accumulation of water, while the amount of differential attenuation observed in the second signal but not the first signal can be at least partially attributed to the presence or accumulation of water. In other embodiments, the first and second signals are not compared to each other directly, but rather changes in attenuation in the first signal are compared to changes in attenuation in the second signal. More specifically, when assessing a first signal where attenuation is not meaningfully affected by the presence of EVLW, the measured attenuation for a given patient position can retain stable over time regardless of changes in EVLW. However, for a second signal where attenuation is significantly impacted by the presence of EVLW, the measured attenuation for a given patient position may fluctuate depending on the patient's EVLW status. Accordingly, if the attenuation of a second signal compared to a past transmission (e.g., the previous value, a historical average value, an average value from when the patient was in a similar position relative to the receiver, etc.) changes more than the attenuation of the first signal compared to a past transmission, it could indicate the presence of or accumulation of EVLW. In some embodiments, the first signal has a frequency of 15 MHz or below, such as about 6.78 MHz or 13.56 MHz or another frequency at which attenuation by water is not significant. In some embodiments, the second signal has a frequency of at least 100 MHz, such as about 434 MHz, 868 MHz, 915 MHz, or 2.4 GHz or another frequency at which significant attenuation by water occurs. Although described as utilizing two signals, in some embodiments the system 100 utilizes more than two signals, such as three, four, five, six, or more (each of which can have a different frequency) to determine/estimate EVLW.

In some embodiments, the system 100 can include one or more sensors 108 associated with, integrated into, or otherwise functionally coupled to the implanted device 102 or other aspects of the system 100. The sensors 108 can provide additional information leading to additional precision regarding how the patient P (and thus the implanted device 102) is positioned relative to the external device 104. For example, if the external device 104 is located in a pad underneath a mattress, gyroscopes, accelerometers, and/or other positional sensors 108 associated with the implanted device 102 can determine if a patient P is laying on their side, back, or stomach, providing further information regarding the position of the lungs L relative to the electromagnetic radiation transmission path 106 between the implanted device 102 and the external device 104. In another embodiment, time of flight calculations can detect the time elapse between when a signal is transmitted from the external device 104 (which is known) and when a signal is detected by the implanted device 102 (which is measured), or vice versa. Such time-of-flight calculations can provide estimations of the distance between the implanted device 102 and the external device 104, which can be used (directly or through comparison to historical values) to provide further insights into the patient's position, and thus the position of the lungs relative to the signal transmission path. In other embodiments, patient position may be inferred from signal phase, phase shifts, and/or other characteristics. Such embodiments and techniques as those disclosed herein are useful as they can eliminate the need for precise alignment between a transmitter and receiver that is a disadvantage of the reflected remote dielectric sensing methods for evaluation of EVLW.

In some embodiments, the external device 104 can be integrated into or otherwise coupled with a mat positioned on or near a patient's bed, allowing for monitoring over the period of time the patient P is in bed. The system 100 can also include a computing device (not shown) having one or more signal processing algorithms that can be applied to (e.g., analyze) data taken over each in-bed interval of the patient P and over a plurality of such intervals to extract variations due to changes in EVLW, even in the presence of signal artifacts due to patient motion and normal diurnal hydration patterns. Said algorithms may include, but are not limited to, spectral decomposition, time-series analysis, and weighted moving averages. One such algorithm, for example, could be a 7-day moving average of the ratios of received to transmitted power at two different frequencies of electromagnetic energy transmitted over closely spaced paths through the body and environment, as described herein.

In some embodiments, no patient interaction is required gather data to measure or estimate EVLW or changes to EVLW using the system 100, and the data can be gathered in a non-clinical setting such as a personal home of the patient P. In such embodiments, it is anticipated that compliance with data gathering will on average increase relative to conventional methods for measuring EVLW (e.g., as tracked by the number of consecutive days measurements are taken) and it will become practical to have multiple measurements taken over the course of a single day. In some embodiments, measurements related to EVLW can be taken while a patient is asleep, a capability not currently available with conventional techniques. In some embodiments, measurements or estimations of EVLW or changes in EVLW can be utilized to adjust or titrate a medical therapy provided by a pharmaceutical regimen and/or by a therapeutic device implanted in or otherwise in-use by a patient.

In some embodiments, the implanted device 102 is part of a medical device or system for providing a therapy for the patient, in addition to acting as a transmitter and/or receiver for determining EVLW. For example, the implanted device 102 may be a part of an implantable interatrial shunting system. FIG. 2A, for example, shows the conventional placement of an interatrial shunt in the septal wall between the LA and RA. Most conventional interatrial shunts (e.g., shunt 150) involve creating a hole or inserting an implant with a lumen into the atrial septal wall, thereby creating a fluid communication pathway between the LA and the RA. As such, elevated left atrial pressure may be partially relieved by unloading the LA into the RA. In early clinical trials, this approach has been shown to improve symptoms of heart failure.

The implantable interatrial shunting system may also include a number of additional features. FIG. 2B, for example, is a schematic illustration of an interatrial shunting system 200 (“system 200”) configured in accordance with embodiments of the present technology. The system 200 is an implanted device within the patient and includes a shunting element or device 202 defining a lumen 204 therethrough. The shunting element 202 can include a first end portion 203a positionable in the LA and a second end portion 203b positionable in the RA. Accordingly, when implanted in the septal wall S, the system 200 fluidly connects the LA and the RA via the lumen 204. When the system 200 is implanted to treat HFpEF, blood generally flows through the lumen 204 in flow direction F (i.e., from the LA to the RA). Under varying subject conditions, the lumen 204 may enable flow in the opposite direction (i.e., from the RA to the LA), or in both directions as the pressure gradient between chambers alternates.

The shunting element 202 can be stabilized in position through forces applied by aspects of the system (e.g., by a flow control mechanism 250, described in greater below) to regions of tissue (e.g., a septal wall) and/or be secured in place by one or more anchoring element(s). For example, the system 200 can include one or more first anchoring elements 220a positioned on the LA side of the septal wall S and/or one or more second anchoring elements 220b positioned on the RA side of the septal wall S (collectively referred to as anchoring elements 220). The first anchoring elements 220a may engage a portion of the septal wall S facing the LA and the second anchoring elements 220b may engage a portion of the septal wall S facing the RA. In some embodiments, the anchoring elements 220 do not make direct contact with a tissue wall. In some embodiments, the anchoring elements 220 extend from and/or are integral with aspects of the shunting element 202. This may be a direct connection via a process such as welding, via an adhesive, or via another connection mechanism known to those skilled in the art. Alternatively, the shunting element 202 and the anchoring elements 220 may be comprised of a single structure, such as a unitary structure composed of a superelastic alloy (e.g., nitinol), and treated so each portion of the device takes on the desired shape. In various embodiments, a connecting element such as a strut or arm may be used to connect the anchoring elements 220 to a horizontal body portion of the shunting element 202. This horizontal body portion may be transseptal, partially-transseptal, or may lie predominantly on one side of the septal wall S, and may have a generally tubular shape that defines the lumen 204. In some embodiments, the system 200 includes a stent-like structure that includes the anchoring elements 220 and an outer frame portion (not shown) that directly interfaces with the septal wall S.

In some embodiments, the shunting element 202 is anchored in place using one or more anchoring elements positioned on only one side of the septal wall S. In yet other embodiments, the system 200 does not include first and second anchoring elements 220a and 220b and the shunting element 202 is secured in place by its general shape, by exerting a radially outward pressure, by another component of the system 200, and/or by other suitable mechanisms.

As with the system 100 described above with respect to FIG. 1, the system 200 may also be configured to use electromagnetic energy transmitted between one or more implanted electronic components of the system 200 and a component external to the patient to monitor EVLW or changes in EVLW. As discussed previously, an electromagnetic signal that passes through a medium can become attenuated (i.e., its signal strength reduced in magnitude). Because the location of the implanted component(s) of the system 200 can be known with respect to lungs L of the patient P, the transmission path for an electromagnetic radiation signal, and therefore the attenuating media through which the signal passes, is at least partially known.

The system 200, for example, can include one or more energy receiving components 230 and one or more energy storage components 232. As discussed in greater detail below, in addition to the system 200 being configured to monitor EVLW or assess changes in EVLW as signal(s) are transmitted along a transmission path between the implanted component(s) and an external device (e.g., external device 104 (FIG. 1)), the system 200 can be further configured to (i) receive energy from an energy source positioned external to a patient's body, and/or (ii) generate energy when exposed to a magnetic or electric field generated by the energy source positioned external to the implanted components of the system (e.g., generated by a source external to the patient's body, generated by a catheter inside the patient's body, etc.). In some embodiments, the energy receiving component 230 can be configured to receive energy transmitted in the radiofrequency (RF) frequency range, including in the high frequency RF range (e.g., between 3-30 MHz) and/or the ultra-high frequency RF range (e.g., 300-3,000 MHz). In other embodiments, the energy receiving component 230 can be configured to receive magnetic or other forms of energy (e.g., heat). The energy receiving component 230 can be a metallic coil, wire, or other antenna, and may be composed at least in part of a high conductivity metal such as copper, silver, or composites thereof. In some embodiments, the energy receiving component 230 may be a generally circular loop or coil of multiple loops coaxial with the lumen 204. In other embodiments, the energy receiving component 230 may be an oval or other non-circular loop or coil of multiple loops bent around the lumen 204. Another embodiment may include a combination of the foregoing loop or coil of multiple loops configured to couple to an external magnetic field regardless of orientation. In some embodiments, a portion of the shunt structure (e.g., anchor elements 220) may serve as all or part of the coil or antenna.

The energy storage components 232 can be configured to store energy received and/or generated by the energy receiving component 230. The energy storage components 232 can include a battery, a supercapacitor, a capacitor, and/or other suitable elements that can retain energy. As described below, the energy received by the energy receiving component 230 and/or stored within the energy storage components 232 can be used (i) to actuate a flow control mechanism 250 to adjust a geometry of the lumen 204 (and/or a geometry of the lumen orifice), thereby altering the flow of blood through the lumen 204, (ii) to power one or more active components of the system 200, such as sensors 240 described below, and/or (iii) for other operations requiring an energy input.

In some embodiments, the electromagnetic energy transmitted from the external device 104 to the implanted device 102 can be used to recharge the energy storage component (or to directly power an active component of the implanted device 102) in addition to being used to assess EVLW.

In some embodiments, the system 200 includes more than one energy receiving component 230. For example, the system 200 can include a first energy receiving component and a second energy receiving component. The first energy receiving component and the second energy receiving component can both be in electrical communication with the energy storage component 232. The first energy receiving component can be configured to receive energy from one or more external sources configured to interface or otherwise communicate with the first energy receiving component. For example, the one or more external sources can include an energy source positioned external to the body and configured to deliver energy remotely to the energy receiving component and/or a catheter that docks or otherwise interfaces with the energy receiving component. The second energy receiving component can be configured to receive energy from the energy storage component 232. Accordingly, energy can be transferred from an external source to the first energy receiving component, from the first energy receiving component to the energy storage component, and from the energy storage component to the second energy receiving component. In some embodiments, the second energy receiving component can be part of the flow control mechanism 250 described below. In some embodiments, a component may simultaneously and/or alternatingly serve as both an energy receiving component and an energy storage component.

The first energy receiving component may be a combination of conductive (e.g., wire or PCB monopole or dipole antenna) and dielectric (e.g., dielectric rod antenna) elements capable of extracting energy from an electromagnetic field, or a conductive element (e.g., wire coil) capable of extracting energy from an AC magnetic field. In some embodiments, the first energy receiving component has substantially no temperature rise when it receives energy, but the second energy receiving component does have a temperature rise when it receives energy. In such embodiments, the first energy receiving component may receive energy from an external source and store it without meaningful dissipation (e.g., dissipation of less than 10%, 15%, or 20% of the total received energy), and later transfer it to the second energy receiving component which dissipates it intentionally. For example, the second energy receiving component may include a temperature sensitive shape memory alloy material that can transition between various configurations when heat is applied. In some embodiments, the first energy receiving component and second energy receiving component can receive energy via pulses occurring at different frequencies. For example, the first energy receiving component can receive energy delivered at a first frequency, and the second energy receiving component can receive energy delivered at a second frequency different than the first frequency. In some embodiments, the second energy receiving component receives a direct current signal. In some embodiments, the first energy receiving component receives an alternating current signal. In some embodiments, the second energy receiving component may directly receive AC energy from an external source. One or more of the electrical components (e.g., the energy receiving components or the energy storage components) can extend along an axial length of the septal implant lumen.

Accordingly, the septal implants described herein can receive energy from an external source (such as external source 104, FIG. 1) and store the energy on the implant in an energy storage component. Upon selective activation, the energy storage component releases the energy in discrete portions. The discrete portions can be defined by the amount of energy released and/or the time period of energy release (e.g., 200 ms or less). In some embodiments, the energy may be selectively released to more than one second energy receiving component and/or to more than one location on the second energy receiving component. In some embodiments, an energy storage component may be pre-loaded with energy and therefore not be configured to receive energy from an external source. For example, the energy storage component can be fully charged or substantially fully charged when implanted in the patient.

In some embodiments, the system 200 includes more than one energy storage component 232. For example, the system 200 can include a first energy storage component and a second energy storage component. In some embodiments, the first energy storage component is “energy dense” and the second energy storage component is “power dense.” The term “energy dense” refers to the amount of energy in a given mass or volume, while the term “power dense” refers to the amount of power in a given mass or volume. In embodiments in which the first energy storage component is energy dense, the first energy storage component can be a battery. In embodiments in which the second energy storage component is power dense, the second energy storage component can be a capacitor. Moreover, in embodiments having more than one energy storage component, one of the energy storage components (e.g., the first energy storage component) can be a primary, non-rechargeable component and another of the energy storage components (e.g., the second energy storage component) can be a secondary, rechargeable component. Furthermore, at the time when the system 200 is implanted, the first energy storage component (e.g., the battery) can be at or near its full stored energy capacity and the second energy storage component (e.g., the capacitor) can be substantially devoid of stored energy. In such embodiments, the second energy storage component can be charged after the implant procedure. In some embodiments, the second energy storage component can be charged using an energy source positioned external to the body. In other embodiments, the second energy storage component can be charged using invasive charging mechanisms, such as a catheter coupled to a power source. In such embodiments, the catheter can dock or otherwise interface with one or more implanted aspects of the system 200 to charge the second energy storage component.

In some embodiments, the energy storage component 232 can be charged (initially charged, recharged, etc.) using an energy source positioned external to the implanted device, for example a source positioned external to the patient. In some embodiments, the charging is conducted directly. In alternative embodiments, the charging is conducted by electrically connecting the energy storage component 232 to the energy receiving component 230, which captures energy from the external source, converts it to an appropriate form, and provides it to the energy storage component 232. In some embodiments the energy storage component 232 may be a secondary (rechargeable) cell (battery), such as a Lithium-Polymer or Lithium-Ion cell. In various embodiments the energy storage component 232 can be a supercapacitor (double electric-layer capacitor). In various embodiments the energy storage component 232 can be a combination of a supercapacitor and conventional capacitor, such as an aluminum electrolytic capacitor, tantalum electrolytic capacitor, or multilayer ceramic capacitor. In some embodiments, the energy receiving component 230 may receive AC magnetic or electromagnetic energy from an external energy source. The received AC energy may be converted to DC using synchronous or non-synchronous (diode) rectification. In some embodiments, the converted DC energy may be boosted and regulated to a level suitable for use by an implanted processor and other implanted electronics via a boost, buck-boost, SEPIC, Zeta, charge pump, or other switch-mode power conversion circuit. In various embodiments, the externally generated magnetic or electromagnetic field can be modulated by the energy source to encode data for transmission to the implanted electronics. In various embodiments, the load presented by the implanted electronics may be modulated to convey data to the external equipment generating the magnetic or electromagnetic field. In various embodiments, NFC (nearfield communications) techniques may be used to implement energy and/or data transfer.

In some embodiments, the one or more energy storage components 232 can be coupled to or otherwise interface with the anchoring elements 220. For example, the energy storage component 232 can interface with a surface of the septal wall S, and the anchoring elements 220 can interface with the energy storage component 232 such that the anchoring element 220 do not directly engage the septal wall S.

The system 200 can also include one or more sensors (e.g., a first sensor 240a, a second sensor 240b, etc.; collectively referred to as the sensors 240). The sensors 240 can be configured to measure one or more physiologic parameters related to the system 200 or the environment proximate to the sensors 240, such as local blood pressure (e.g., LA blood pressure, RA blood pressure, etc.), flow velocity, pH, SpO2, SpC, SpMet, heart rate, cardiac output, myocardial strain, etc. In some embodiments, the sensors 240 can also include one or more features similar to the sensor(s) 108 of the system 100 (FIG. 1).

The sensors 240 can be, for example, (1) embedded in an implantable component of the system 200, (2) implanted yet spaced apart from other implantable components of the system 200, and/or (3) included on a wearable patch or device external to the body. If included on a wearable patch or device, the wearable patch or device could provide power to the sensor (e.g., RFID/NFC). In some embodiments, the wearable patch or device can also read sensor data. The sensors can be continuously recording or can be turned on at select times.

In one embodiment, the first sensor 240a is a pressure sensor positionable within the LA and the second sensor 240b is a pressure sensor positionable within the RA. In some embodiments, the system 200 can further include a processor (not shown) configured to calculate a pressure differential between the LA and the RA based on information measured by the sensors 240 or other information. As described below, the system 200 may be adjusted based on the parameters measured by the sensors 240 and/or the pressure differential or other information calculated by the processor.

In some embodiments, the sensors 240 may be configured as pressure sensors. For example, the pressure sensor can include a cavity covered by a membrane, where the membrane communicates with a strain sensing element, an element that varies the frequency of a resonant circuit, and/or other elements that vary with the deflection of the membrane and alter an electrically measurable quantity. The membrane may be in direct contact with a measurement region, conformally coated with a material directly in contact with a measurement region, and/or enclosed in a rigid vessel filled with a fluid communicating with a membrane that is in contact with a measurement region, where the fluid may be a liquid such as silicone oil or a gas such as air. Embodiments with a sensor or a conformally coated sensor directly in contact with a measurement region will additionally incorporate a means of communicating pressure information to electronics enclosed in a housing.

The system 200 also includes a flow control mechanism 250 (e.g., an actuation mechanism, a flow control assembly, an actuation assembly, etc.). The flow control mechanism 250 is configured to selectively change a geometry or other characteristic of the shunting element 202 and/or the lumen 204 to change the flow of fluid through the lumen 204. For example, the flow control mechanism 250 can be configured to selectively increase a diameter of the lumen 204 (or lumen orifice) and/or selectively decrease a diameter of the lumen 204 (or lumen orifice) in response to an input. In other embodiments, the flow control mechanism 250 is configured to otherwise affect a geometry of the lumen 204. Accordingly, the flow control mechanism 250 can be coupled to the shunting element 202 and/or can be included within the shunting element 202. For example, in some embodiments the flow control mechanism 250 is part of the shunting element 202 and at least partially defines the lumen 204. In other embodiments, the flow control mechanism 250 is spaced apart from but operably coupled to the shunting element 202.

In some embodiments, at least a portion of the flow control mechanism 250 can comprise a shape memory material. The shape memory portion can include nitinol, a nitinol-based alloy, a shape memory polymer, a pH-based shape memory material, or any other suitable material configured to move or otherwise adjust as would be understood by one of skill from the description herein. The shape memory portion can be characterized by a curve that defines the amount of deformation the portion undergoes in response to a particular input (e.g., an applied stress). For example, the flow control mechanism 250 can include a nitinol element that is configured to change shape in response to exposure to energy, such as heat. In such embodiments, the flow control mechanism 250 can be selectively actuated by applying energy directly or indirectly to the nitinol element. Additional features and examples of flow control mechanisms incorporating one or more shape memory components are described in International Patent Application No. PCT/US2020/049996, filed Sep. 9, 2020, the disclosure of which is incorporated by reference herein in its entirety and for all purposes.

In some embodiments, the flow control mechanism 250 can be actuated using energy stored in the energy storage component 232. Accordingly, rather than directly applying energy to the flow control mechanism 250, a clinician can use a controller (described below) to actuate the flow control mechanism 250 (thereby adjusting the geometry of the shunting element 202 and/or the lumen 204) using energy stored in the energy storage component 232. This permits the clinician to decouple the process of (1) applying energy to the energy receiving component 230, and (2) adjusting the shunting element 202. Accordingly, the energy storage component 232 may store energy for a period of time (e.g., minutes, hours, days, months, etc.) and, upon a determination that the flow through the shunting element 202 should be changed, a user can direct the energy storage component 232 to release stored energy and direct it to one or more aspects of the flow control mechanism 250. In other embodiments, the system 200 can automatically direct the energy storage component 232 to release stored energy and direct it the flow control mechanism 250 to adjust a flow through the shunting element 202.

In one embodiment, the energy storage component 232 can be configured to discharge energy (e.g., in the form of a discharge pulse) to heat an actuation element of the flow control mechanism 250. For example, the energy storage component 232 may discharge energy to one or more actuation elements that are composed of a metallic material such that applying energy to the metallic material leads to resistive heating, inductive heating, or both. The metallic material can be a shape memory material such as nitinol that has been manufactured such that the resistive heating results in at least a partial transition of the material from a first material phase or state (e.g., martensitic phase, R-phase, etc.) to a second material phase or state (e.g., an R-phase, an austenitic phase, etc.). If the shape memory actuation element is deformed relative to its preferred geometry (e.g., manufactured geometry, original geometry, heat-set geometry, shape-set geometry, etc.), transitioning the shape memory actuation element from the first material phase to the second material phase can induce a geometric change in the shape memory actuation element to and/or toward its preferred geometry. The heat can therefore be applied to the one or more shape memory actuation elements to affect a property of said component (e.g., a length, width, position, stiffness, etc.). The movement of the shape memory actuation element can result in a change of the shape or dimension of the lumen 204. In some embodiments, the heated element (e.g., the shape memory actuation element) is different than the mechanism that moves to change the geometry of the shunting element 202 and/or the lumen 204. For example, the actuation element may be mechanically connected to connecting features that translate the movement of the actuation element into a change in a feature (e.g., a change in size, shape, etc.) of a different component of the device (e.g., a horizontal lumen component).

In some embodiments, the energy storage component 232 and/or the energy receiving component 230 can be omitted and flow can be adjusted by directly applying energy to the flow control mechanism 250. In such embodiments, a portion of the flow control mechanism 250 can be configured to receive energy (e.g., heat, light, RF, ultrasound, microwave, etc.) from an energy source positioned external to the body (e.g., an RF transmitter) and, in response to the received energy, adjust the flow through the lumen 204. For example, the flow control mechanism 250 can include a heat activated shape memory element, and adjusting the lumen 204 via the flow control mechanism 250 can comprise heating the shape memory element to change the geometry of the shape memory alloy element, thereby adjusting flow through the shunting element 202, as previously described.

In some embodiments, the flow control mechanism 250 is coupled to a processor (not shown) that calculates the pressure differential between the LA and RA based, at least in part, on the measurements taken by the sensors 240. If the calculated pressure differential falls outside of a predetermined range, the processor can direct the flow control mechanism 250 to change the flow through the shunting element 202. In some embodiments, the sensors 240, the processor, and the flow control mechanism 250 operate in a closed-loop system to adjust the shunting element 202. In other embodiments, the pressure differential sensed by the sensors 240 is transmitted to a display external to the patient, and a user (e.g., a clinician) adjusts the flow through the shunting element 202 based at least in part on the measured pressure differential. In such embodiments, the physician may adjust the flow using a non-invasive energy source (e.g., an RF transmitter) and/or by interfacing with a controller 260 (described below).

As provided above, the system 200 can include a controller 260 connectable to or integrated with one or more implanted aspects of the system 200. Suitable controllers include, for example, mobile device applications, computers, dedicated controllers, etc. The controller 260 can connect to various implanted aspects of the system 200 via WiFi, Bluetooth (e.g., BLE 5.0), electromagnetic, ultrasound, RF, or other wireless means. Alternatively, the controller 260 may be coupled to implanted aspects via a wired connection. The controller 260 provides a user interface such that a user (e.g., the patient, a physician, etc.) can selectively control the system 200 via the controller. For example, a physician can input a desired flow rate, pressure/pressure gradient, or other input, and the controller 260 can communicate (either directly or indirectly) with the flow control mechanism 250 such that the flow control mechanism 250 manipulates the shunting element 202 to achieve a desired flow rate and/or flow resistance through the shunting element 202.

Some embodiments of the present technology adjust the geometry of the shunting element 202 and/or the lumen 204 consistently (e.g., continuously, hourly, daily, etc.). Consistent adjustments might be made, for example, to adjust the flow of blood based on an exertion level and/or heart rate of the patient, which changes frequently over the course of a day. For example, the system 200 can have a baseline state in which the lumen 204 is substantially closed and does not allow substantial blood flow between the LA and RA, and an active state in which the lumen 204 is open and allows blood to flow between the LA and RA. The system 200 can transition from the baseline state to the active state whenever the exertion level (e.g., as measured by the heart rate) of the patient increases due to exercise, stress, or other factors. In another embodiment, consistent adjustments can be made based on, or in response to, sensed physiological parameters, including, for example, sensed LA pressure and/or RA pressure via sensors 240. If the LA pressure increases, the system 200 can automatically increase a diameter of the lumen 204 between the LA and the RA and allow increased blood flow. In another example, the system 200 can be configured to adjust based on, or in response to, an input parameter from another device such as a pulmonary arterial pressure sensor, insertable cardiac monitor, pacemaker, defibrillator, cardioverter, wearable, external ECG or PPG, and the like.

Additional suitable implantable interatrial shunting systems and associated methods are described in International Patent Application No. PCT/US2020/063360, filed Dec. 3, 2020, the disclosure of which is incorporated by reference herein in its entirety and for all purposes.

Although the foregoing describes transmitting electromagnetic energy between an implanted device and an external device, in some embodiments the electromagnetic energy may be transmitted between a plurality of implanted devices, or between a plurality of implanted devices and an external device. In such embodiments, the electromagnetic energy transmitted between a plurality of implanted devices can charge an energy storage component of the receiving implanted device in addition to being used to assess EVLW.

### Examples

Several aspects of the present technology are set forth in the following examples:

1. A system for measuring extravascular lung water (EVLW) in a patient, the system comprising:
a first device configured to be implanted in the patient, the first device further configured to (a) transmit electromagnetic energy, (b) receive electromagnetic energy, or (c) both (a) and (b); and
a second device configured to be external to the patient, the second device further configured to (d) receive electromagnetic energy transmitted by the first device, (e) transmit electromagnetic energy to the first device, or (f) both (d) and (e),
wherein an electromagnetic energy transmission path between the first device and the second device passes through the patient's lungs, and
wherein the system is configured to determine EVLW in the patient based, at least in part, on an attenuation or phase shift of electromagnetic energy transmitted between the first device and the second device along the electromagnetic energy transmission path.

2. The system of example 1 wherein the system is configured to transmit a first electromagnetic energy signal at a first frequency at which substantial signal attenuation by water does not occur, and wherein the system is configured to transmit a second electromagnetic energy signal at a second frequency at which substantial signal attenuation by water does occur.

3. The system of example 2 wherein the first frequency is less than 15 MHz, and wherein the second frequency is greater than 100 MHz.

4. The system of examples 2 or 3 wherein the first electromagnetic energy signal and the second electromagnetic energy signal are both transmitted along the electromagnetic energy transmission path.

5. The system of any of examples 2-4 wherein the system is configured to determine EVLW in the patient based, at least in part, on a ratio of attenuation of the first electromagnetic energy signal to the second electromagnetic energy signal.

6. The system of any of examples 1-5, further comprising one or more sensors configured to determine a position of the patient, wherein the determined position is used in part to determine the EVLW of the patient.

7. The system of example 6 wherein the one or more sensors are carried by the first device.

8. The system of any of examples 1-7 wherein the first device does not move relative to the patient's lungs.

9. The system of any of examples 1-8 wherein the first device is an interatrial shunt.

10. A method of measuring extravascular lung water (EVLW) in a patient, the method comprising:
transmitting a first electromagnetic signal having a first frequency along a transmission path between a first device and a second device, wherein at least one of the first device and the second device is implanted in the patient, and wherein, when transmitted along the transmission path, at least a portion of the first electromagnetic signal passes through patient lung tissue;
transmitting a second electromagnetic signal having a second frequency different than the first frequency along the transmission path between the first device and the second device, wherein, when transmitted along the transmission path, at least a portion of the second electromagnetic signal passes through the patient lung tissue;
determining an attenuation of the first electromagnetic signal and the second electromagnetic signal when transmitted along the transmission path; and
based at least in part on the attenuation of the first electromagnetic signal and the second electromagnetic signal, estimating EVLW in the patient.

11. The method of example 10 wherein the first electromagnetic signal is not substantially attenuated by water by virtue of the first frequency, and wherein the second electromagnetic signal is attenuated by water by virtue of the second frequency.

12. The method of example 10 or 11 wherein the first frequency is less than 15 MHz, and wherein the second frequency is greater than 100 MHz.

13. The method of any of examples 10-12 wherein estimating EVLW in the patient based at least in part on the attenuation of the first and second electromagnetic signals includes comparing the magnitude of attenuation of the first electromagnetic signal to the magnitude of attenuation of the second electromagnetic signal along the transmission path.

14. The method of any of examples 10-13 wherein the steps of transmitting the first electromagnetic signal, transmitting the second electromagnetic signal, and determining the attenuation are iteratively repeated, and wherein estimating the EVLW includes comparing change in attenuation of the second electromagnetic signal along the transmission path over time to change in attenuation of the first electromagnetic signal along the transmission path over time.

15. The method of any of examples 10-14 wherein the first and second electromagnetic signals are transmitted simultaneously.

16. The method of any of examples 10-15 wherein the first device is external to the patient and the second device is implanted in the patient, and wherein the first and second electromagnetic signals are transmitted from the first device toward the second device.

17. The method of example 16 wherein the first device is positioned adjacent the patient's bed.

18. The method of example 16 wherein the second device includes an interatrial shunt.

### CONCLUSION

Embodiments of the present disclosure may include some or all of the following components: a battery, supercapacitor, or other suitable power source; a microcontroller, FPGA, ASIC, or other programmable component or system capable of storing and executing software and/or firmware that drives operation of an implant; memory such as RAM or ROM to store data and/or software/firmware associated with an implant and/or its operation; wireless communication hardware such as an antenna system configured to transmit via Bluetooth, WiFi, IEEE 802.15.4, or other protocols known in the art; energy harvesting means, for example a coil or antenna which is capable of receiving and/or reading an externally-provided signal which may be used to power the device, charge a battery, initiate a reading from a sensor, or for other purposes. Embodiments may also include one or more sensors, such as pressure sensors, impedance sensors, accelerometers, force/strain sensors, temperature sensors, flow sensors, optical sensors, cameras, microphones or other acoustic sensors, ultrasonic sensors, ECG or other cardiac rhythm sensors, SpO2 and other sensors adapted to measure tissue and/or blood gas levels, blood volume sensors, and other sensors known to those who are skilled in the art. Embodiments may include portions that are radiopaque and/or ultrasonically reflective to facilitate image-guided implantation or image guided procedures using techniques such as fluoroscopy, ultrasonography, or other imaging methods. Embodiments of the system may include specialized delivery catheters/systems that are adapted to deliver an implant and/or carry out a procedure. Systems may include components such as guidewires, sheaths, dilators, and multiple delivery catheters. Components may be exchanged via over-the-wire, rapid exchange, combination, or other approaches.

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A system for measuring extravascular lung water (EVLW) in a patient, the system comprising:
a first device configured to be implanted in the patient, the first device further configured to (a) transmit electromagnetic energy, (b) receive electromagnetic energy, or (c) both (a) and (b); and
a second device configured to be external to the patient, the second device further configured to (d) receive electromagnetic energy transmitted by the first device, (e) transmit electromagnetic energy to the first device, or (f) both (d) and (e),
wherein an electromagnetic energy transmission path between the first device and the second device passes through the patient's lungs, and
wherein the system is configured to determine EVLW in the patient based, at least in part, on an attenuation or phase shift of electromagnetic energy transmitted between the first device and the second device along the electromagnetic energy transmission path.

2. The system of claim 1 wherein the system is configured to transmit a first electromagnetic energy signal at a first frequency at which substantial signal attenuation by water does not occur, and wherein the system is configured to transmit a second electromagnetic energy signal at a second frequency at which substantial signal attenuation by water does occur.

3. The system of claim 2 wherein the first frequency is less than 15 MHz, and wherein the second frequency is greater than 100 MHz.

4. The system of claim 2 wherein the first electromagnetic energy signal and the second electromagnetic energy signal are both transmitted along the electromagnetic energy transmission path.

5. The system of claim 2 wherein the system is configured to determine EVLW in the patient based, at least in part, on a ratio of attenuation of the first electromagnetic energy signal to the second electromagnetic energy signal.

6. The system of claim 1, further comprising one or more sensors configured to determine a position of the patient, wherein the determined position is used in part to determine the EVLW of the patient.

7. The system of claim 6 wherein the one or more sensors are carried by the first device.

8. The system of claim 1 wherein the first device does not move relative to the patient's lungs.

9. The system of claim 1 wherein the first device is an interatrial shunt.

10. A method of measuring extravascular lung water (EVLW) in a patient, the method comprising:
transmitting a first electromagnetic signal having a first frequency along a transmission path between a first device and a second device, wherein at least one of the first device and the second device is implanted in the patient, and wherein, when transmitted along the transmission path, at least a portion of the first electromagnetic signal passes through patient lung tissue;
transmitting a second electromagnetic signal having a second frequency different than the first frequency along the transmission path between the first device and the second device, wherein, when transmitted along the transmission path, at least a portion of the second electromagnetic signal passes through the patient lung tissue;
determining an attenuation of the first electromagnetic signal and the second electromagnetic signal when transmitted along the transmission path; and
based at least in part on the attenuation of the first electromagnetic signal and the second electromagnetic signal, estimating EVLW in the patient.

11. The method of claim 10 wherein the first electromagnetic signal is not substantially attenuated by water by virtue of the first frequency, and wherein the second electromagnetic signal is attenuated by water by virtue of the second frequency.

12. The method of claim 10 wherein the first frequency is less than 15 MHz, and wherein the second frequency is greater than 100 MHz.

13. The method of claim 10 wherein estimating EVLW in the patient based at least in part on the attenuation of the first and second electromagnetic signals includes comparing the magnitude of attenuation of the first electromagnetic signal to the magnitude of attenuation of the second electromagnetic signal along the transmission path.

14. The method of claim 10 wherein the steps of transmitting the first electromagnetic signal, transmitting the second electromagnetic signal, and determining the attenuation are iteratively repeated, and wherein estimating the EVLW includes comparing change in attenuation of the second electromagnetic signal along the transmission path over time to change in attenuation of the first electromagnetic signal along the transmission path over time.

15. The method of claim 10 wherein the first and second electromagnetic signals are transmitted simultaneously.

16. The method of claim 10 wherein the first device is external to the patient and the second device is implanted in the patient, and wherein the first and second electromagnetic signals are transmitted from the first device toward the second device.

17. The method of claim 16 wherein the first device is positioned adjacent the patient's bed.

18. The method of claim 16 wherein the second device includes an interatrial shunt.
